(19) United States
(12) Patent Application Publication
(10) Pub. No.: US 2009/0197906 A1
(43) Pub. Date: Aug. 6, 2009

(54) REVERSION OF MALIGNANT PHENOTYPE WITH 9-HYDROXY ELLIPTICINE DERIVATIVES

(76) Inventors: Christian Auclair, Paris (FR); Valerie Polard, Sainte Genevieve Des Bois (FR); Andrei Maksimenko, Brunoy (FR)

Correspondence Address: STITES & HARBISON PLLC, 1199 NORTH FAIRFAX STREET, SUITE 900, ALEXANDRIA, VA, 22314, US

(21) Appl. No.: 12/301,534

(22) PCT Filed: May 21, 2007
(86) PCT No.: PCT/IB07/01307

§ 371 Date: Nov. 19, 2008

Related U.S. Application Data
(60) Provisional application No. 60/838,860, filed on Aug. 21, 2006.

(30) Foreign Application Priority Data
May 22, 2006 (EP) 06290822.3

(51) Int. Cl. A61K 31/44; C07D 471/00

(52) U.S. Cl. 514/285; 546/70

(57) ABSTRACT

The invention relates to the use of 9-hydroxy ellipticine derivatives for the treatment of cancer. 9-hydroxy ellipticine derivatives may prove particularly useful for the treatment of metastatic cancers or cancers escaping conventional cytotoxic chemotherapies.

This application claims benefit to U.S. Provisional application No. 60/838,860 filed on Aug. 21, 2006, the disclosure of which is incorporated herein by reference in its entirety as if fully set forth herein.

The invention relates to the use of 9-hydroxy ellipticine derivatives for the treatment of cancer. These 9-hydroxy ellipticine derivatives may prove particularly useful for the treatment of metastatic cancers or cancers escaping conventional cytotoxic chemotherapies.

In non cancer cells, adhesion to the extracellular matrix and to neighbouring cells plays a central role in the control of cell survival, growth, differentiation and motility (K. A. Beningo et al., J. Cell Biol. 153 (2001), pp. 881-888; S. M. Frisch and R. A. Screaton, Curr. Opin. Cell Biol. 13 (2001), pp. 555-562 and F. M. Watt, EMBO J. 21 (2002), pp. 3919-3926). Upon oncogenic transformation, profound changes occur in cell morphology and the organization of the cytoskeleton, in cell motility and in growth factor- or adhesion-dependent cell proliferation (for a review, see G. Pawlak and D. M. Helfman, Curr. Opin. Genet. Dev. 11 (2001), pp. 4147). Disruption of the actin cytoskeleton and a concomitant reduction in the number of focal adhesions are common features accompanying cell transformation induced by various oncogenes. That the actin cytoskeleton plays a fundamental role in oncogenesis is suggested by the association of anchorage-independent growth and tumorigenicity with the rearrangements of the actin filament network observed in transformed cells (P. Kahn et al., Cytogenet. Cell Genet. 36 (1983), pp. 605-611). Adhesive interactions involve specialized transmembrane receptors that are linked to the cytoskeleton through junctional plaque proteins (for a review, see Nagafuchi, Curr. Opin. Cell Biol. 13 (2001), pp. 600-603). The synthesis of several actin-binding proteins, including α-actinin, vinculin, tropomyosin and profilin, is down-regulated in transformed cells and overexpressing these proteins in tumor cells suppresses the transformed phenotype, which allows them to be considered as tumor suppressors.

Ellipticine is a natural plant alkaloid product which was isolated from the evergreen tree of the Apocynaceae family, and which has the formula (I)

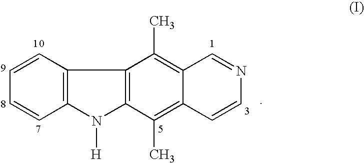

Ellipticine was found to have cytotoxic and anticancer activity (Dalton et al., Aust. J. Chem., 1967. 20, 2715).

The ellipticine derivative hydroxylated in position 9 (9-hydroxyellipticinium) was found to have greater antitumoural activity than ellipticine on many experimental tumours (Le Pecq et al., Proc. Natl. Acad, Sci., USA, 1974, 71, 5078-5082) but was found to display a limited activity for the treatment of human cancers (Le Pecq et al., Cancer Res., 1976, 36, 3067).

Researches were performed to identify an ellipticine derivative appropriate for human therapeutics and lead to the preparation of Celiptium, or N2-methyl-9-hydroxyellipticinium (NMHE), which has been used for the treatment of some human cancers, in particular for the treatment of bone metastasis of breast cancers. A series of compounds derived from 9-hydroxy ellipticine were thus developed and had formula (II)

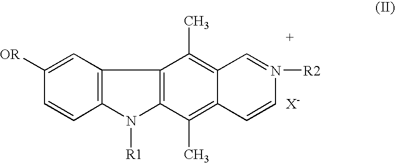

wherein R and R1 are hydrogen or an alkyl group, and R2 is an alkyl group optionally substituted, and X⁻ is a quaternizing anion. These compounds have been described in the U.S. Pat. No. 4,310,667.

The planar polycyclic structure of these compounds was found to interact with DNA through intercalation. Furthermore, these compounds were found to be implicated in multiple modes of action, including DNA binding, generation of oxidative oxygen species and modification of enzyme function; most notably that of topoisomerase II and telomerase (see for instance Auclair, 1987, Archives of Biochemistry and Biophysics, 259, 1-14).

Pharmacologically, a number of toxic side effects have been shown to be problematic. In particular Celiptium was found to induce renal toxicity. However, some ellipticine derivatives, such as 2-(diethylamino-2-ethyl)9-hydroxyellipticinium-chloride (Auclair et al., 1987, Cancer Research, 47, 6254-6261), were found to have improved safety and anticancer activities in animals. Albeit the improved properties of 2-(diethylamino-2-ethyl)9-hydroxyellipticinium-chloride made it selected for phase I trial, the development of this compound was then abandoned.

Other 9-hydroxy ellipticine derivatives, such as 2-(diethylamino-2-ethyl)9-hydroxyellipticinium acetate, 2-(diisopropylamino-ethyl)9-hydroxyellipticinium acetate and 2-(beta piperidino-2-ethyl)9-hydroxyellipticinium, had been described for instance in the U.S. Pat. No. 4,310,667.

The development of drugs effective against human cancers and having limited toxic side effects remains a critical need. The challenge is in particular to succeed in identifying anticancer drugs acting mainly through a non-cytotoxic process. In this field of investigation, the inventors hypothesised that changes in cell phenotype, and more specifically in the cytoskeletal architecture, which is one of the main molecular mechanisms underlying tumor progression, could be a pertinent target process.

The inventors have unexpectedly demonstrated that a limited number of 9-hydroxy ellipticine derivatives have anticancer activity which is mediated by a non-cytotoxic process (i.e. non directly linked to biological damages in cells) inducing actin network rearrangement, thereby inducing phenotypic reversion of tumor cells thanks to the rescue of adhesion and motility control. Moreover, phenotypic reversion is obtained with non-cytotoxic concentrations, i.e. concentrations which have no significant effect on both cell proliferation and cell survival.

Thus, the 9-hydroxy ellipticine derivatives identified by the inventors provide anticancer drugs acting mainly through a non-cytotoxic process.

### Ellipticine Derivatives

The 9-hydroxy ellipticine derivatives identified as inducing malignant phenotypic reversion at non-cytotoxic concentrations have the formula (III):

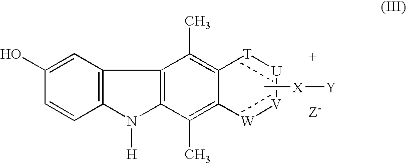

optionally in the form of an acid addition salt,

wherein

X is an alkyl group having 2 or 3 carbon atoms, optionally branched, and optionally substituted by OH, NRR′, CN, OR, COOR, wherein R and R′ are independently H or a C1-C4 alkyl group;

Y is —NR1R2, wherein R1 and R2 are independently H or a C1-C6 alkyl group, or R1 and R2 form together with the N atom, to which they are attached, a saturated or unsaturated 5- or 6-membered heterocycle, wherein —NR1R2 may be in the form of a quaternary ammonium salt resulting from the addition of a pharmaceutically acceptable mineral or organic acid, so that the compound of formula (I) is in the form of an acid addition salt;

or Y is a benzyl, a phenyl or a C5 or C6 aryl or 5- or 6-heteroaryl group

Z⁻ is an anion of a pharmaceutically acceptable mineral or organic acid;

the -X-Y side chain is attached to either T, U, V or W as appropriate;

T, U, V and W are either a C atom or a N atom, so as to form a pyridyl ring and the remaining T, U, V and/or W are C atoms,

provided that the -X-Y side chain is attached to the one of T, U, V and W being a N atom,

it being understood that [symbol] represents either a single bond or a double bond, as appropriate, so that the system formed with the fused pyridyl ring is aromatic and the resulting cation

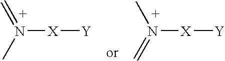

is formed.

According to an embodiment, the 9-hydroxy ellipticine derivatives of the invention have the formula (IV):

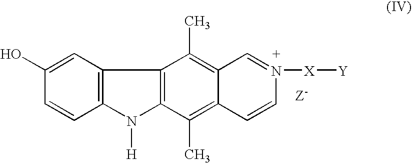

wherein X is an alkyl group having 2 or 3 carbon atoms, optionally branched, and optionally substituted by OH, NRR′, CN, OR, COOR wherein R and R′ are independently H or a C1-C4 alkyl group;

Y is —NR1R2, wherein R1 and R2 are independently H or a C1-C6 alkyl group, or N, R1 and R2 optionally form together a saturated or unsaturated 5- or 6-membered heterocycle, wherein —NR1R2 may be in the form of a quaternary ammonium salt resulting from the addition of a pharmaceutically acceptable mineral or organic acid, so that the compound of formula (I) is in the form of an acid addition salt;

or Y is a benzyl, a phenyl or a C5 or C6 aryl or 5- or 6-heteroaryl group; and

Z⁻ is an anion of a pharmaceutically acceptable mineral or organic acid.

As used herein, “alkyl” means an aliphatic hydrocarbon group which may be straight or branched having about 1 to about 20 carbon atoms in the chain. Preferred alkyl groups have 1 to about 12 carbon atoms in the chain, still preferably 1 to 6 carbon atoms. Branched means that one or lower alkyl groups such as methyl, ethyl or propyl are attached to a linear alkyl chain. <<Lower alkyl>> means about 1 to about 4 carbon atoms in the chain which may be straight or branched. The alkyl may be substituted with one or more <<alkyl group substituants>> which may be the same or different, and include for instance halo, cycloalkyl, hydroxy, alkoxy, amino, acylamino, aroylamino, carboxy.

“Aryl” means an aromatic monocyclic or multicyclic ring system of about 5 to about 14 carbon atoms, preferably of about 6 to about 10 carbon atoms. The aryl is optionally substituted with one or more substituents, which may be the same or different, and are as defined herein. Exemplary aryl groups include phenyl or naphthyl, or phenyl substituted or naphthyl substituted.

As used herein, the term “heteroaryl” refers to a 5 to 14, preferably 5 to 10 membered aromatic hetero, mono-, bi- or multicyclic ring, which is formed by removal of one hydrogen atom. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazoyl, tetrazolyl, isoquinolyl, benzothienyl, isobenzofuryl, pyrazolyl, carbazolyl, benzimidazolyl, isoxazolyl, etc.

“Pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

A pharmaceutically acceptable mineral or organic acid may be selected from the group consisting of hydrochloric, hydrobromic, hydroiodic, sulphuric, phosphoric, hexafluorophosphoric, nitric, carbonic, citric, salicylic, methanesulfonic, acetic, oxalic, maleic, fumaric, succinic, tartric, aspartic, glutamic, lactic, malonic, benzoic, cyclohexansulfamic, and cinnamic acids. (See, for example S. M. Berge, et al., <<Pharmaceutical Salts,>> J. Pharm. Sci., 66: p. 1-19 (1977)). In the above general formulae (III) and (IV):
Z⁻ is the corresponding single charged anion deriving from the above acid.

Preferably, in the above formulae (III) and (IV), Z⁻ is methanesulfonate (also called mesylate, CH₃SO₃⁻); and additionally
—NR1R2 may be in the form of a quaternary ammonium salt resulting from the addition of a pharmaceutically acceptable mineral or organic acid as defined above, preferably the methanesulfonic acid, so that the compound of formula (I) may bear two positive charges.

In the above 9-hydroxy ellipticine derivatives, X is preferably ethyl or propyl.

Where Y is an aryl group, Y may be advantageously selected from the group consisting of pyridine and pyrimidine,

Where Y is —NR1R2, advantageously, each of R1 and R2 may be an ethyl group, or Y may be a piperidine or a pyrrolidine group.

According to certain embodiments, X is ethyl and Y is selected from the group consisting of diethylamino, pyrrolidinyl, benzyl, phenyl, piperidine, pyridine and pyrimidine.

According to certain embodiments also, X is propyl and Y is selected from the group consisting of diethylamino, pyrrolidinyl, benzyl, phenyl, piperidine, pyridine and pyrimidine.

Preferred 9-hydroxy ellipticine derivatives are as follows:

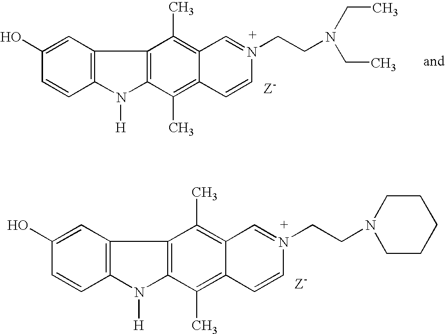

and their resulting quaternary ammonium salts,

where Z⁻ is chosen from the above single charged anions.

More specifically, for the use of the invention, the 9-hydroxy ellipticine derivative may be 2-(diethylamino-2-ethyl)-9-hydroxyellipticinium chloride, 2-(diethylamino-2-ethyl)-9-hydroxyellipticinium methanesulfonate, 2-(beta piperidino-2-ethyl)-9-hydroxyellipticinium chloride, 2-(beta piperidino-2-ethyl)-9-hydroxyellipticinium methanesulfonate and their resulting quaternary ammonium salts.

Furthermore, preferred 9-hydroxy ellipticine derivatives are 2-(diethylamino-2-ethyl)-9-hydroxyellipticinium methanesulfonate, 2-(beta piperidino-2-ethyl)-9-hydroxyellipticinium chloride, and 2-(beta piperidino-2-ethyl)-9-hydroxyellipticinium methanesulfonate and their resulting quaternary ammonium salts.

More preferably, the 9-hydroxy ellipticine derivative according to the invention

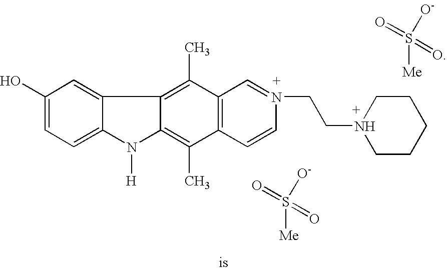

Methods of preparing 9-hydroxy ellipticine derivatives have been described for instance in the U.S. Pat. No. 4,310,667.

### Methods of Treatment

The above 9-hydroxy ellipticine derivatives induce remodeling of the actin cytoskeleton in tumor cells, thereby leading to decreased cell motility and recovery of cell adhesion. This process leads in vivo to selective apoptosis of tumor cells resulting from various mechanisms including eventually from an immune response of the host possibly involving TCL toxic effect.

Thus, the invention relates to the use of a 9-hydroxy ellipticine derivative formula (III) or (IV) for the manufacture of a medicament intended for the treatment of cancer. The invention also relates to a method of treating cancer, by reversing the transformed phenotype of a tumor cell, comprising administering to a subject in need thereof a therapeutically effective amount of a 9-hydroxy ellipticine derivative as defined above. However, in this use and method, it may be preferred that the 9-hydroxy ellipticine derivative is not 2-(diethylamino-2-ethyl)-9-hydroxyellipticinium chloride, 2-(diethylamino-2-ethyl)-9-hydroxyellipticinium acetate, 2-(diisopropylamino-ethyl)-9-hydroxyellipticinium acetate, or 2-(beta piperidino-2-ethyl)-9-hydroxyellipticinium acetate.

The invention further relates to the use of a 9-hydroxy ellipticine derivative of formula (III) or (IV) for the manufacture of a medicament intended for reversing the transformed phenotype of a tumor cell. The invention also relates to a method of reversing the transformed phenotype of a tumor cell, comprising administering to a subject in need thereof a therapeutically effective amount of a 9-hydroxy ellipticine derivative as defined above.

As used herein, the term “subject” denotes a mammal, such as a rodent, a feline, a canine, and a primate. Preferably a subject according to the invention is a human.

In the context of the invention, the term “treating” or “treatment”, as used herein, means reversing, alleviating, inhibiting the progress of, or preventing the disorder or condition to which such term applies, or one or more symptoms of such disorder or condition.

A “therapeutically effective amount” refers to an amount of compound sufficient to result in amelioration of a symptom of a particular disorder or disease. Advantageously, the method of treatment of the invention may be implemented using non-cytotoxic amounts of 9-hydroxy ellipticine derivative, i.e. concentrations which have no significant effect on both cell proliferation and cell survival.

As used herein, the term “transformed phenotype” denotes a change which may occur (i) in cell morphology, and/or (ii) in the organization of the cytoskeleton, and/or (iii) in cell motility and/or (iv) in growth factor- or adhesion-dependent cell proliferation. Said transformed phenotype is a hallmark of tumor cells.

Examples of changes in cell morphology include cells displaying a more rounded shape, fewer cytoplasmic extensions, reduced spreading area, and reduced cell/cell contacts. A change in the organization of the cytoskeleton may be in particular a disruption of the actin cytoskeleton, which is typically associated with a concomitant reduction in the number of focal adhesions.

“Reversing the transformed phenotype of tumor cells” means making the tumor cells to recover the phenotype of a normal (i.e. non-tumoral) cell. Reversal of the transformed phenotype by 9-hydroxy ellipticine derivatives is in particular induced by actin network rearrangement.

Reversal of the transformed phenotype may be assessed by the one skilled using methods of assay readily known in the art.

These methods include for instance:
a semi-solid or soft agar growth assay (clonogenicity assay);
a cell motility assay;
a method of measurement of stationary polymerized actin in a lysate of cells, as described in the international patent application WO 2004/057337. This method comprises of an index of tumor aggressivity. Briefly the method comprises lysing cells under non denaturing conditions, adjusting the total proteins concentration of the lysate, adding fluorescently labelled actin monomers and components necessary for polymerization of endogenous actin (e.g. ATP), and measuring polymerized actin;
an assessment of morphological changes and cytoskeleton organisation of cells by actin, zyxin, actinin, or B-catenin labelling following microscopy observation according to conventional procedures.

The medicament or method according to the invention induces selective apoptosis of tumor cells and thereby provides a non-cytotoxic method of treatment of cancer.

According to the invention, the tumor cell may be a cell originating from any tumor, e.g. a primary or metastatic tumor, a solid tumor or soft tissue tumor, or a leukemia. Examples of solid or soft tumor cells include bladder, breast, bone, brain, cervical, colorectal, endometrial, kidney, liver, lung, nervous system, ovarian, prostate, testicular, thyroid, uterus, pancreas and skin cancer cells. Leukaemias include for instance chronic myeloproliferative diseases, myelodysplastic syndromes, acute non lymphocytic leukaemias, B-cell acute lymphocytic leukaemias, T-cell acute lymphocytic leukaemias, non Hodgkin lymphomas, and chronic lymphoproliferative diseases.

Tumor cells which are expected to be most responsive to the 9-hydroxy ellipticine derivatives are those characterized by an invasive phenotype associated with cytoskeleton breakdown, increased cell motility and/or decreased cell-cell adhesion, as may be observed in aggressive sarcoma and during epithelium-mesenchymal transition occurring in early step of metastasis.

It is an advantage of the invention that pursuant to their capacity to reverse the malignant phenotype of a cell, the 9-hydroxy ellipticine derivatives as described herein constitute true anti-invasive agents. Hence, according to an embodiment, the tumor cell is a metastatic cell. Accordingly, the medicament or method according to the invention may be intended for the treatment of metastasis.

Furthermore, the 9-hydroxy ellipticine derivatives as defined herein have anticancer activity which is mediated by a non cytotoxic process. These compounds may advantageously be administered to treat cancer in a subject escaping conventional cytotoxic chemotherapies with inhibitors of DNA replication such as DNA binding agents in particular alkylating or intercalating drugs, antimetabolite agents such as DNA polymerase inhibitors, or topoisomerase I or II inhibitors, or with anti-mitogenic agents such as alkaloids. These cytotoxic compounds include for instance actinomycin D, adriamycin, bleomycine, carboplatin, cisplatin, chlorambucil, cyclophosphamide, doxorubicin, etoposide, 5-fluorouracil, 6-mercaptopurine melphalan, methotrexate, paclitaxel, taxotere, vinblastine, and vincristine.

As used herein, the term “subject escaping cytotoxic chemotherapy” denotes in particular subjects in which cytotoxic chemotherapy does not modify tumor progression.

One or more 9-hydroxy ellipticine derivatives, as defined herein, may be administered simultaneously or consecutively to the subject to be treated.

Moreover, the 9-hydroxy ellipticine derivatives may be administered in combination (i.e. simultaneously or consecutively) with a differentiating agent, in particular with vitamin A, its synthetic analogs, and metabolites (retinoids), vitamin D or its analogs, or peroxisome proliferator-activated receptors (PPAR) ligands.

Retinoids may be for instance all-transretinoic acid (ATRA), N-(4-hydroxyphenyl) retinamide (4HPR), 13-cis-retinoic acid (13-CRA), or 9-cis-retinoic acid (9-CRA).

Vitamin D or its analogs include in particular 25-dihydroxyvitamin D3 (1,25-(OH)2 D3), which is the dihydroxylated metabolite normally formed from vitamin D3, or 1alpha.-hydroxy-vitamin D3, 1alpha.-hydroxyvitamin D2, 1alpha-hydroxyvitamin D5, fluorinated vitamin D derivatives.

PPAR ligands are in particular PPARα or PPARγ activators. Selective PPARγ agonists include classic TZDs (troglitazone, rosiglitazone, pioglitazone, and ciglitizone; see Forman et al., 1995, Cell, 83:803-812; Lehmann et al., 1995, J. Biol. Chem. 270:12953-12956) and non-TZD-type agonists. Representatives of the latter include N-(2-benzoylphenyl)-L-tyrosine derivatives, such as GW 1929, GI 262570, and GW 7845, which are among the most potent and selective PPARγ agonists identified to date (see Henke et al., 1998, J. Med. Chem., 41:5020-5036; Cobb et al., 1998, J. Med. Chem., 41:5055-5069). GW 0207, a 2,3-disubstituted indole-5-carboxylic acid, is also a potent and selective PPARγ agonist (Henke et al., 1999, Bioorg. Med. Chem. Lett., 9:3329-3334). Fibrates or farnesol are example of PPARα agonists.

Therefore, the 9-hydroxy ellipticine derivatives useful according to the invention may also be mixed another therapeutic compound to form pharmaceutical compositions (with or without diluent or carrier) which, when administered, provide simultaneous administration of a combination of active ingredients resulting in the combination therapy of the invention. In particular the invention provides a pharmaceutical composition comprising a 9-hydroxy ellipticine derivative of formula (III) or (IV) and a differentiating agent, as are defined above.

Further to a simultaneous administration, the 9-hydroxy ellipticine derivatives useful according to the invention may also be administered separately or sequentially with another therapeutic compound, in particular a differentiating agent as defined above. Thus the invention further provides a product comprising a 9-hydroxy ellipticine derivative of formula (III) or (IV), and a differentiating agent, as a combined preparation for simultaneous, separate or sequential use for the treatment of cancer, in particular for reversing the transformed phenotype of a tumor cell.

While it is possible for the 9-hydroxy ellipticine derivatives to be administered alone it is preferably to present them as pharmaceutical compositions. The pharmaceutical compositions, both for veterinary and for human use, useful according to the present invention comprise at least one 9-hydroxy ellipticine derivatives, as above defined, together with one or more pharmaceutically acceptable carriers and optionally other therapeutic ingredients.

In certain preferred embodiments, active ingredients necessary in combination therapy may be combined in a single pharmaceutical composition for simultaneous administration.

As used herein, the term “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The pharmaceutical compositions can be administered in a suitable formulation to humans and animals by topical or systemic administration, including oral, rectal, nasal, buccal, sublingual, vaginal, parenteral (including subcutaneous, intramuscular, intravenous, intradermal, intrathecal and epidural), intracisternal and intraperitoneal. It will be appreciated that the preferred route may vary with for example the condition of the recipient.

The formulations can be prepared in unit dosage form by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Total daily dose of the 9-hydroxy ellipticine derivatives administered to a subject in single or divided doses may be in amounts, for example, of from about 0.001 to about 100 mg/kg body weight daily and preferably 0.01 to 10 mg/kg/day, still preferably 0.01 to 1 mg/kg/day, in particular 0.1 to 1 mg/kg/day, or 1 to 10 mg/kg/day. Examples of daily dosages are 0.05 mg/kg, 0.125 mg/kg, 0.25 mg/kg, 0.5 mg/kg, 1 mg/kg, 1.25 mg/kg, 2.5 mg/kg, 5 mg/kg, and 10 mg/kg. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

The invention will be further illustrated in view of the following examples.

### FIGURES

FIG. 1 shows the structure of BA016DD537 (2-(β-piperidinoethyl)-9-hydroxyellipticinium chloride).

FIG. 2 is a representation of the time course of actin stabilization by BA016DD537 in NIH 3T3 EF extract. BA016DD537 was added at zero time with polymerization buffer and NIH 3T3 EF extract. Reaction mixtures contained BA016DD537 at concentrations symbolized as follows: 100 nM BA016DD537 (▴), 200 nM BA016DD537 (♦), control malignant NIH 3T3 EF cells (▾), control normal NIH 3T3 cells (▪). Data represent mean standard deviation; n=3.

FIG. 3 is a representation of the time course of actin stabilization by 100 nM BA016DD537 (♦), 200 nM BA016CA107 (□) and 200 nM BA016CA77 (*) in NIH 3T3 EF extract. The drugs were added at zero time with polymerization buffer and NIH 3T3 EF extract. Control malignant NIH 3T3 EF cells (Δ), control normal NIH 3T3 cells (◯) are also shown. Data represent mean standard deviation; n=3.

FIG. 4 is a fluorescence microscopy examination of actin fibers in NIH 3T3 EF cells, treated or not with BA016DD537, and compared with control NIH 3T3 cells. BA016DD537 increases the actin fibres in transformed NIH 3T3 EF cells. Normal and malignant NIH 3T3 cells were analysed by in situ immunofluorescence by using FITC-phalloidin to visualize the actin filaments and Dapi to visualize the nucleus. (A) Control malignant NIH 3T3 EF cells; (B) Control normal NIH 3T3 cells; (C) Malignant NIH 3T3 EF cells treated with 100 nM BA016DD537; (D) Malignant NIH 3T3 EF cells treated with 200 nM BA016DD537.

FIG. 5 shows the morphological changes of MIA PaCa-2 cells treated by BA016FZ539 (2-(beta piperidino-2-ethyl)-9-hydroxyellipticinium methanesulfonate). A: Control, B: Cells treated by 4 μM of BA016FZ539 for 3 days (×200).

FIG. 6 displays proliferation test of B16BL6 cells treated with BA016DD537 (2-(beta piperidino-2-ethyl)-9-hydroxyellipticinium chloride) in the presence or absence of 13CRA and ATRA. The concentration of retinoic acids used was fixed at 10 nM.

### EXAMPLES

### Example 1

### Modulation of Actin Dynamics

Actin dynamics is known to be impaired in tumor cells with a subsequent decrease of F-actin to G-actin ratio. Actin dynamics has been quantified in tumor cell extracts using the fluorescence anisotropy assay which gains access to rate constant of F-actin elongation (k) and steady state concentration of F-actin (Δ mA).

Materials and Methods:

All reactions were carried out at 22° C. and fluorescence anisotropy signal was recovered at 520 nm with excitation at 490 nm in a Beacon 2000 (Panvera). Alexa 488 actin (Molecular Probes) was centrifuged at 35 000 rpm for 2 h at 4° C. to sediment residual actin polymers in a Beckman L5-50B ultracentrifuge. The fluorescence remaining in the supernatant was considered to be likely due to monomers or small actin filaments (5-10 monomers) that do not pellet under conditions described previously. 80% of the supernatant was withdrawn; the concentration was defined through fluorescence measurements (excitation at 490 nm and signal recovering at 520 nm). The ultracentrifuged actin concentration was calculated using the non ultracentrifuged Alexa 488 actin as a standard. The supernatant was aliquoted, frozen in liquid nitrogen and stored at −80° C.

Before experiment, an aliquot of ultracentrifuged Alexa 488 actin was diluted to a concentration of 1 mg/ml in G buffer (5 mM Tris pH 8.1, 2 mM CaCl₂, 0.2 mM DTT, 0.2 mM ATP). 3 μl of diluted Alexa 488 actin was mixed in 168 μl of G buffer and actin monomers anisotropy was measured before the addition of 4 μl of polymerisation buffer (2.5 M KCl, 50 mM MgCl₂, 25 mM ATP), 5 μl of G buffer in the presence or in the absence of the chemical molecule and 20 μl cellular extract of normal NIH 3T3 cells or malignant NIH 3T3 EF cells at 2 mg/ml. The final concentration of Alexa 488 actin was 4 nM. The ratio of unlabelled to labelled actin was about 140/4 nM. Measurements were made each 10 sec for 200 sec. Actin monomers anisotropy value was subtracted, yielding the anisotropy enhancement (Δ mA). The data were fitted with the equation Y=Ymax. [1−exp(−K.X)]. The curves start at zero and ascend to Ymax that corresponds to the steady state anisotropy value (Δ mA eq), with a rate constant K. Y is anisotropy values of which monomers anisotropy is subtracted and X is the time.

Results:

The effects of BA016DD537 on these parameters are as follows:

TABLE 1
Effects of BA016DD537 concentration on anisotropy
enhancement in NIH 3T3 EF extracts

| | Normal | Malignant | Malignant NIH 3T3 EF | Malignant NIH 3T3 EF |
|---|---|---|---|---|
| | NIH 3T3 | NIH 3T3 EF | cells in the presence of | cells in the presence of |
| | cells | cells | BA016DD537 at 100 nM | BA016DD537 at 200 nM |
| Δ mA | 59.46 | 40.47 | 52.38 | 61.17 |
| k · sec⁻¹ | 0.1225 | 0.0960 | 0.1636 | 0.1737 |

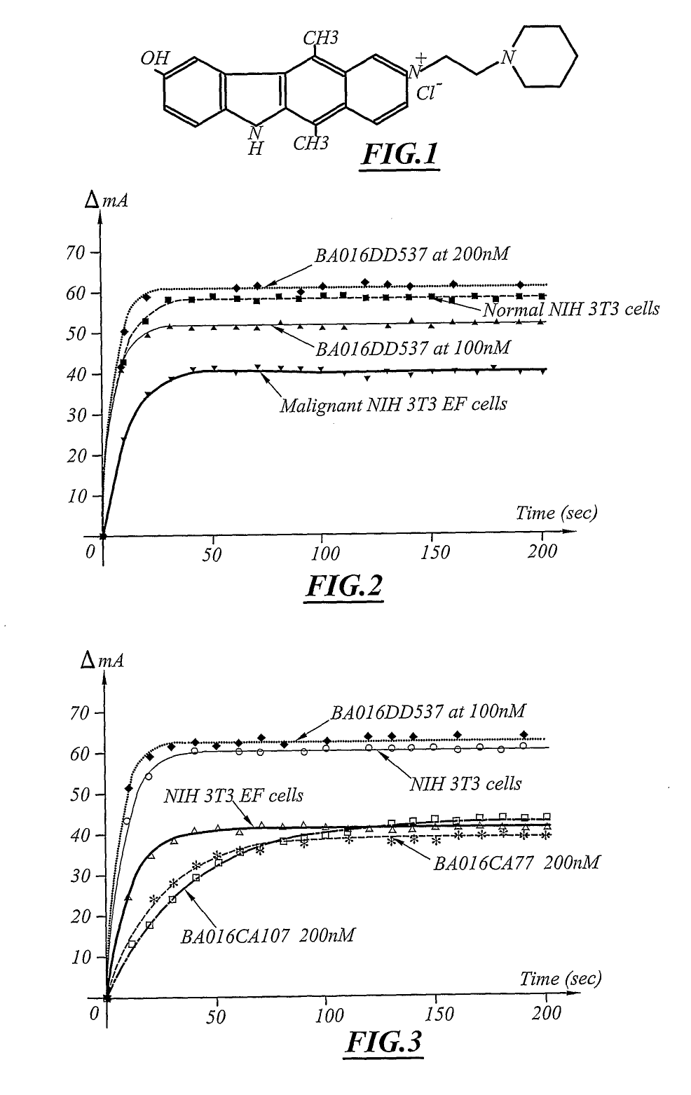

An anisotropy enhancement was observed for NIH 3T3 EF extract in the presence of the 2-(β-piperidinoethyl)-9-hydroxyellipticinium chloride (BA016DD537) as compared with anisotropy measured in the absence of BA016DD537 (FIG. 2).

NIH 3T3 EF cells display, as compared to native NIH 3T3 cells, lower pseudo first order rate constant of actin elongation as well as a lower amount of F-actin at the steady state. It was therefore assumed that cytosolic fractions prepared from NIH 3T3 EF cells are convenient materials to screen molecules which may modulate actin dynamics, including those which could preferentially bind to actin filaments such as BA016DD537. When added to assay medium, BA016DD537 increases the actin-F elongation rate constant and the actin-F steady state value. FIG. 2 shows typical kinetics observed following the addition of increasing concentrations of BA016DD537. In the presence of 200 nM of BA016DD537, the actin dynamics of the NIH 3T3 EF cytosolic fractions is similar to those observed using NIH 3T3 cytosolic fractions. Thus, the BA016DD537 may be used as actin polymerisation promoting agent.

### Example 2

### Modulation of Actin Dynamics and Ex Vivo Inhibition of Cell Motility by 9-hydroxy-2(beta-ethyl)-ellipticinium Acetate (BA016CA107) and 9-hydroxy-2(beta-methyl)-ellipticinium Acetate (Celiptium, BA016CA77)

Celiptium was known as anti-cancer drug. The mechanism of action of the 9-hydroxy-2(beta-ethyl)-ellipticinium acetate (BA016CA107) and Celiptium (BA016CA77) were compared with that of BA016DD537 by steady state fluorescence anisotropy measurement assay (materials and methods, example 1). Their ability to inhibit the cells motility was also investigated (materials and methods, example 4).

Results:

As shown in FIG. 3, BA016CA77 and BA016CA107 do not increase the actin-F elongation rate constant and the actin-F steady state value. In the presence of 200 nM of BA016CA77 or BA016CA107, the actin dynamics of the NIH 3T3 EF cytosolic fractions is similar to those observed using NIH 3T3 EF cytosolic fractions without treatment. Thus, BA016CA77 and BA016CA107 cannot be used as actin polymerisation promoting agents.

TABLE 2
Effects of BA016DD537, BA016CA77 and BA016CA107 concentration
on anisotropy enhancement in NIH 3T3 EF extracts

| | | | Malignant NIH | Malignant NIH | Malignant NIH |
|---|---|---|---|---|---|
| | Normal | | 3T3 EF cells in | 3T3 EF cells in | 3T3 EF cells in |
| | NIH | Malignant | the presence of | the presence of | the presence of |
| | 3T3 | NIH 3T3 | BA016DD537 at | BA016CA107 at | BA016CA77 at |
| | cells | EF cells | 200 nM | 200 nM | 200 nM |
| ΔmA | 59.46 | 40.47 | 61.17 | 42.14 | 38.33 |
| k · sec⁻¹ | 0.1225 | 0.0960 | 0.1737 | 0.0280 | 0.0416 |

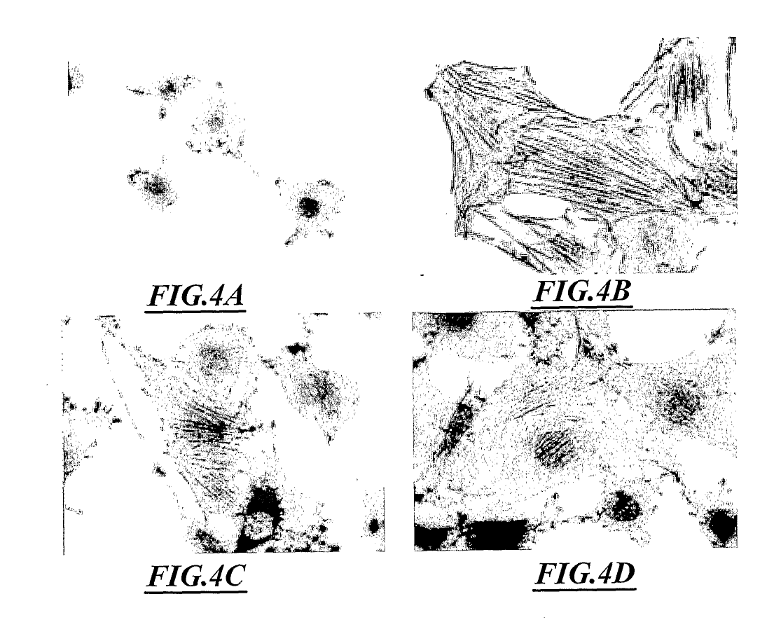

The behavior of malignant cells treated by BA016CA77 and BA016CA107 drugs was also compared to that of non-treated malignant cells in a wound healing assay. Treated malignant cells were found to migrate beyond the border of the wound into its whole area (FIG. 4). The cells treated with drugs at non cytotoxic concentrations migrate in the same way as non treated malignant cells.

In conclusion, neither 9-hydroxy-2(beta-ethyl)-ellipticinium acetate, nor 9-hydroxy-2(beta-methyl)-ellipticinium acetate (Celiptium) were found active thus indicating that the nature of the side chain in position 2 plays a critical role in their ability to modulate actin dynamics.

### Example 3

### Rescue of F-Actin Network in Tumor Cells

Material and Methods:

Malignant NIH 3T3 EF cells were seeded onto glass cover slips at a density of 2000 cells per cm². The next day, BA016DD537 was applied to NIH 3T3 EF cells at various non cytotoxic concentrations (100 nM and 200 nM). Three days later, cells were fixed for 10 min in PBS containing 3.7% formaldehyde at 4° C. before examination with a fluorescence microscope. The formaldehyde solution was neutralized with 50 mM NH₄Cl. Extraction was carried out for 4 min with 0.4% Triton X-100 in PBS. Cells were incubated for 1 h with blocking buffer (3% bovine serum albumin in PBS) and then for 20 min with FITC-phalloidin (Sigma) at room temperature. Cover slips were mounted in Vectashieldk (Zymed) and observed through a fluorescence microscope (Nikon).

Results:

The drug BA016DD537 is able to rebuilt actin network in tumor cells at non cytotoxic concentration as shown in FIG. 4. NIH 3T3 EF cells, treated with non cytotoxic BA016DD537 concentration, recover a morphology close to that of NIH 3T3 cells: cells are spread, possess numerous intercellular contacts and actin cytoskeleton is well organized in a stress fibres network.

In similar experimental conditions, neither 9-hydroxy-2-ethyl)-ellipticinium acetate, nor 9-hydroxy-2(methyl)-ellipticinium acetate (Celiptium) were found active.

### Example 4

### Ex Vivo Inhibition of Cell Motility

Tumor cell invasion and metastasis, later points in cancer progression clearly involve cell motility. The central engine of cell movement, as well as cell shape change in general, is the cytoskeleton and the key component of the cytoskeleton involved in animal cell locomotion is actin. Consequently, actin dynamics modulation may result in cell motility impairment which in turn should restrain invasion and metastasis.

For these reasons, BA016DD537 has been tested in a cell motility assay.

Material and Methods:

Wound healing assay was performed to evaluate the effect of BA016DD537 on motility of malignant NIH 3T3 EF cells and melanoma cell lines B16F10 and B16BL6. All the cells were incubated at 37° C. in a humidified 5% CO2 atmosphere. About 100 000-200 000 cells were seeded in a 6-well culture plate and BA016DD537, at the different concentrations, was added 24 hours later. Cells were grown for 3 days to confluence about 90-95% and small scratch-wounds (about 200 μm-1 mm width) were made with a pipette tip. Cell debris were removed, then the cultures were incubated in complete medium for 10 h in the presence of the same concentration of BA016DD537. Then, the cells were fixed for 10 min in PBS containing 3.7% formaldehyde at 4° C. The healing was observed with a phase contrast light microscope using Zeiss software.

Results:

The invasive melanoma cells B16F10 and B16BL6 as well as the tumorigenic NIH-3T3 EF cells expressing the fusion protein EWS-FLI-1 display a high motility phenotype. To get an overall evaluation of their motile properties, we compared the behaviour of drug malignant cells treated by BA016DD537 with that of non-treated malignant cells in a wound healing assay. The non treated malignant cells migrate beyond the border of the wound into its whole area. On the contrary, the malignant cells treated by BA016DD537 do not migrate at all into the wound. BA016DD537 inhibited the malignant cell motility in a dose dependent manner. Cell treatment with BA016DD537 at concentration as low as 50 nM results in the complete inhibition of B16F10 melanoma and NIH 3T3 EF cell migration. Also we observed the inhibition of the B16BL6 cells motility by BA016DD537 at non cytotoxic concentration. Therefore, the effect of the selected BA016DD537 drug is not due to toxic effect.

### Example 5

### Ex Vivo Antiproliferative Effect of 9-Hydroxy Ellipticine Derivatives

Malignant cells display the property to grow on semi-solid medium such as methyl-cellulose according to an anchorage independent manner.

Antitumor activity of 2-(beta piperidino-2-ethyl)-9-hydroxyellipticinium chloride (BA016DD537) and 2-(beta piperidino-2-ethyl)-9-hydroxyellipticinium methanesulfonate (BA016FZ539) related to phenotypic reversion was assessed by the inhibition of colonies formation in semi-solid medium. Several cell lines have been investigated. Inhibition of colony formation was compared to inhibition of cell proliferation as measured by MTT reduction.

Material and Methods:

Cloning Assay

Cells were embedded in complete culture medium supplemented with 0.8% methylcellulose (Methocel MC4000, Sigma), seeded in triplicate into 35-mm dishes (Greiner Bio-one Ref 627102, Dominique Dutscher) and incubated at 37° C. in a humidified 5% CO₂ atmosphere. The number of cells seeded was 1000 cells per dish. After one to three weeks, according to cell lines, macroscopic clones were counted.

MTT Assay

Growth studies were performed using the [3-(4,5 dimethylthiazol-2-yl)-2,5-diphenyl-2H-tetrazolium bromide] (Sigma) calorimetric assay.

About 1500 to 5000 cells, according to the cell line, were seeded in a 96-well culture plate 24 hours before adding increasing concentrations of BA016DD537 or BA016FZ539. The plate was incubated at 37° C. for 3 days. 10 μl MTT stock solution (5 mg/ml in phosphate buffer saline) was added to 90 μl of complete medium in each well, the incubation was continued for 3 h at 37° C. 100 μl of lysis buffer (10% sodium dodecyl sulfate, 1% HCl 1N; pH 4.7) was added to each well, and the plate was incubated overnight. The absorbance was determined using an Integrated EIA Management System (Labsystem) at a wavelength of 570 nm. The proliferation rates were calculated from the OD readings using the untreated cells as 100%.

Typical results obtained are shown in Tables 3 to 6 below.

TABLE 3
Inhibition of colony formation and
cell proliferation by BA016DD537

| | | Inhibition of colony |
|---|---|---|
| | Cytotoxic effect (MTT) | formation (methylcellulose) |
| NIH 3T3 EF | IC50 = 400 nM | IC50 = 30 nM |
| B16F10 | IC50 = 500 nM | IC50 = 35 nM |

TABLE 4
Inhibition of colony formation and cell proliferation by BA016FZ539
on cell lines expressing EWS/FLI-1 proto-oncogen

| | | Inhibition of colony |
|---|---|---|
| | Cytotoxic effect (MTT) | formation (methylcellulose) |
| NIH 3T3 EF | IC50 = 400 nM | IC50 = 30 nM |
| SK-N-MC | IC50 = 840 nM | IC50 = 205 nM |

TABLE 5
Inhibition of human melanoma cell lines colony
formation and cell proliferation by BA016FZ539

| Melanoma | | Inhibition of colony |
|---|---|---|
| cell lines | Cytotoxic effect (MTT) | formation (methylcellulose) |
| B16F10 | IC50 = 500 nM | IC50 = 35 nM |
| B16BL6 | IC50 = 212 nM | IC50 = 29 nM |
| A375 | IC50 = 2.9 μM | IC50 = 97 nM |
| C9 | IC50 = 2.1 μM | IC50 = 132 nM |
| 451 Lu | IC50 = 4.2 μM | IC50 = 2 μM |
| 1205 Lu | IC50 = 1.6 μM | IC50 = 247 nM |
| SKMEL28 | IC50 = 10.7 μM | IC50 = 515 nM |
| HT144 | IC50 = 9 μM | IC50 = 125 nM |

TABLE 6
Inhibition of human pancreas carcinoma cell lines colony
formation and cell proliferation by BA016FZ539

| Pancreas | | Inhibition of colony |
|---|---|---|
| cell lines | Cytotoxic effect (MTT) | formation (methylcellulose) |
| Mia Paca-2 | IC50 = 7.6 μM | IC50 = 640 nM |
| PANC-1 | IC50 = 22 μM | IC50 = 4.3 μM |

BA016DD537 and BA016FZ539 were thus found to display a marked inhibitory activity on colony formation in semi-solid medium. Inhibition of colony formation occurs at non-antiproliferative concentration as measured using the MTT test.

### Example 6

### Antitumor Activities

Antitumor activity against B16 melanoma can be assessed in mice using i.p. graft of malignant cells followed by i.p. treatment. This type of protocol which by-pass various biodisponibility parameters gives information roughly on the maximal antitumor activity which can be expected for a given tumor.

Experimental Protocol:

Melanoma B16 cells (4×10⁵) were injected in B6D2F1 mice using i.p. route at J0. Drugs, dissolved in sterile distilled water (0.5 ml) were injected daily using as well i.p. route from J1 to J9 at various concentrations. Control mice received distilled water only according the same protocol.

Treated and control mice were counted daily. TIC (mean survival of treated mice/mean survival of control mice) was calculated at J9. T/C>125% indicates a significant antitumor activity.

The results of the experiments are summarized in Table 7 below:

TABLE 7
ratio of mean survival of mice treated with Celiptium or
BA016DD537 on mean survival of control mice (T/C ratio)

| Drugs | Dose (mg/kg/inj.) | T/C (%) |
|---|---|---|
| Methyl-2OH-9E acetate | 0.5 | 58 |
| (Celiptium) | 0.25 | 122 |
| | 0.125 | 121 |
| β-pipéridinoéthyl-2 OH-9E | 10 | 87 |
| acetate | 7.5 | 210 |
| (BA016DD537) | 6.25 | 196 |
| | 3.12 | 217 |
| | 1.56 | 168 |
| | 0.78 | 149 |
| | 0.39 | 133 |

Thus BA016DD537 exhibits a marked antitumor activity against B16 melanoma, Optimal dose of 3.12 mg/kg yields a T/C of 217%. The reference drug Celiptium has no significant antitumor activity using this protocol,

### Example 7

### Antimetastatic Activity

The invasive phenotype displayed by B16F10 murine melanoma cells is characterized by the ability of tumor cells to efficiently form metastasis in lung when injected by i.v. route. In order to assess the anti-invasive property, the effect of 2-(beta piperidino-2-ethyl)-9-hydroxyellipticinium methanesulfonate on this process has been tested.

Experimental Protocol:

100 μl of a B16F10 cellular suspension (4.10⁵ cells) were injected using i.v. route into the retro-orbital sinus of the mice. The 2-(beta piperidino-2-ethyl)-9-hydroxyellipticinium methanesulfonate (BA016FZ539) solution was administrated i.v to mice 24 hours and 72 hours after cell injection at a dose of 5 mg/Kg (first experiment) and 7.5 mg/kg (second experiment). In control group, mice were injected iv with physiological serum. Seven days later, the mice were sacrificed, the lungs were excised and metastatic nodules were counted under a dissecting microscope.

TABLE 8
Percentage of inhibition of B16F10 cell
pulmonary metastases by BA016FZ539

| | Dose J1, J3 | Inhibition of pulmonary metastases |
|---|---|---|
| | (mg/kg/inj.) | development (% of control values) |
| Experiment 1 | 5 | 21% (p = 0.0904) |
| Experiment 2 | 7.5 | 39.6% (p = 0.3269) |

In the experimental conditions used, BA016FZ539 displays a significant anti-invasive activity as evidenced by the significant decrease of lung metastasis following i.v. injection of B16F10 melanoma cells.

### Example 8

### In Vitro Antiproliferative Effect of 9-Hydroxy Ellipticine Derivatives on Small Cell Lung Cancer Cell Lines

Antitumor activity of BA016FZ539 (2-(beta piperidino-2-ethyl)-9-hydroxyellipticinium methanesulfonate) was assessed by the inhibition of cell proliferation as measured by the sulforhodamine test.

Three small cell lung cancer cell lines have been investigated: NCI-H510, NCI-H446 and NCI-H187.

Small cell lung cancers (SCLC) account for 15-25% of all lung cancers diagnosed each year (Bonfill et al. 1975-1977 and 1987-1989. Int J Cancer 65: 751-754, 1996). SCLC cell lines can be sub-grouped into 2 major classes; classic SCLC cell lines (NCI-H187 and NCI-H510) which express elevated levels of neuroendocrine markers, and variant SCLC cell lines which fail to express one or more of the neuroendocrine markers.

Some studies have shown that variant cell lines, in contrast to classic lines, are radioresistant in vitro and have increased expression of c-myc oncogene (Carney et al., Cancer Research 45, 2913-2923, Jun. 1985).

Materials and Methods: SRB Assay

Growth studies were performed using the Sulforhodamine B (SRB) calorimetric assay (Sigma).

SRB assay is used for cell density determination based on the measurement of cellular protein content. This method has been optimized for toxicity screening of compounds in adherent cells in a 96-well format (Skehan et al., Proc. Amer. Assoc. Cancer Res. 1989, 30:2436).

About 50 000 NCI-H510, NCI-H446 or NCI-H187 cells were seeded in a 96-well culture plate while adding increasing concentrations of BA016FZ539.

After an incubation period, cell monolayers are fixed with 10% (wt/vol) trichloroacetic acid and stained for 30 min, after which the excess dye is removed by washing repeatedly with 1% (vol/vol) acetic acid. The protein-bound dye is dissolved in 10 mM Tris base solution for optic density determination (OD) at 510 nm using a microplate reader.

The proliferation rates were calculated from the OD readings using the untreated cells as 100%.

The SRB protein stain assay was compared with the tetrazolium (MTT) colorimetric assay for in vitro chemosensitivity testing of various human small cell lung cancer cell lines.

The SRB assay has several advantages over the MTT assay. For example, some compounds can directly interfere with MTT reduction without having any effects on cell viability, while SRB staining is rarely affected by this type of interference. Furthermore, SRB staining is independent of cell metabolic activity.

Results:

TABLE 9
inhibition of cell proliferation by BA016FZ539
measured by SRB or MTT assay

Viability 72 h

| | IC50 (μM) SRB | IC50 (μM) MTT |
|---|---|---|
| NCI-H510 | 5.8 +/− 1.9 | 14.8 |
| NCI-H446 | 22.4 +/− 6.7 | 9.8 +/− 0.2 |
| NCI-H187 | 16.9 +/− 6.8 | 7.9 |

(Mean +/− SEM)

In Table 9, it can be observed that variant SCLC cell line (NCI-H446) shows a better resistance to BA016FZ539 than classic SCLC cell lines (NCI-H510 and NCI-H187).

### Example 9

### In Vitro Antiproliferative Effect of 9-Hydroxy Ellipticine Derivatives on Pancreatic Cancer Cell Lines

Antitumor activity of BA016FZ539 (2-(beta piperidino-2-ethyl)-9-hydroxyellipticinium methanesulfonate) was assessed by the inhibition of cell proliferation as measured by the sulforhodamine test.

Two pancreatic cancer cell lines have been investigated: MIA PaCa-2 and PANC-1.

Materials and Methods:

The effects BA016FZ539 on the growth of MIA PaCa-2 and PANC-1 pancreatic cells were tested over a range of concentrations from 500 μM to 0.16 μM and measured by using the SRB calorimetric assay.

About 5000 MIA PaCa-2 or PANC-1 cells were seeded in a 96-well culture plate while adding increasing concentrations of BA016FZ539.

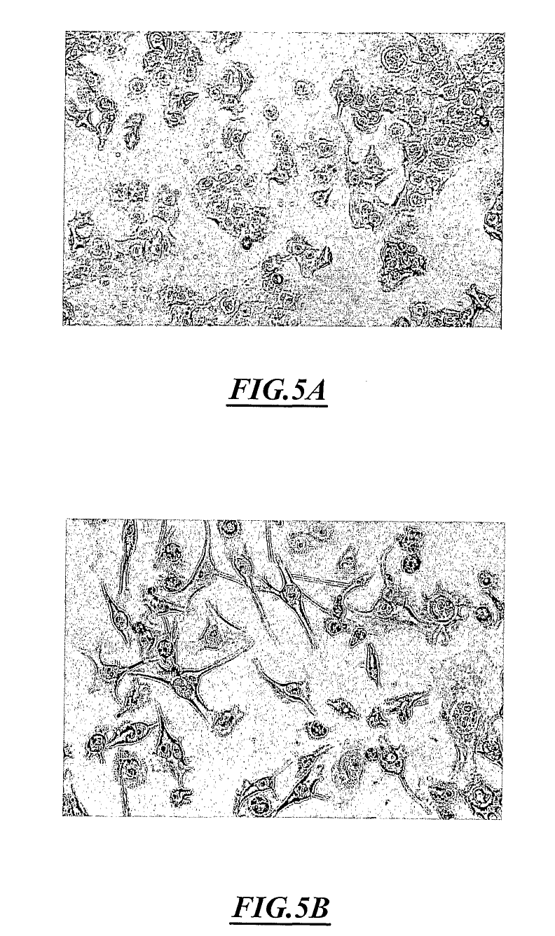

Changes in configuration and number of pancreatic cell lines were observed under microscopic observation (FIG. 5).

Results:

TABLE 10
Inhibition of cell proliferation by BA016FZ539

Viability 72 h

| | IC50 (μM) SRB | IC50 (μM) MTT |
|---|---|---|
| MIA PaCA-2 | 10.24 +/− 3.2 | 7.58 +/− 0.3 |
| PANC-1 | 20.68 +/− 2.9 | 22.02 |

(Mean +/− SEM)

FIG. 5 shows the reversal from the transformed phenotype (FIG. 5, A) to a normal phenotype (FIG. 5, B) by BA016FZ539 in MIA PaCa-2 cell line.

This effect was observed only in MIA PaCa-2 cell line and not in PANC-1 cell line. Reversal of the transformed phenotype is here associated with changes in cell morphology including more cytoplasmic extensions and an increase of cell spreading area. Thus, BA016FZ539 exhibits a better antitumor activity against MIA PaCA-2 cell line than PANC-1 cell line (Table 10).

In conclusion, the data presented here show that the BA016FZ539 exerts multiple antitumoral effects on human cancer cell lines. BA016FZ539 was found to significantly inhibit cell growth in SCLC and pancreatic cell lines with IC50 between 6 and 20 μM. These results suggest also the capacity to reverse the malignant phenotype of a pancreatic cell line, MIA PaCa-2. These cells treated by BA016FZ539 exhibit morphological changes suggesting a modification in cytoskeleton organization.

### Example 10

### Ex Vivo Inhibition of Cell Motility

The 9-hydroxy ellipticine derivatives may be administered in combination with a differentiating agent, in particular with vitamin A, its synthetic analogs, and metabolites (retinoids), vitamin D or its analogs. Retinoids may be for instance all-transretinoic acid (ATRA), N-(4-hydroxyphenyl) retinamide (4HPR), 13-cis-retinoic acid (13CRA), or 9-cis-retinoic acid (9CRA).

In this example the efficacy of combinations of BA016DD537 (2-(beta piperidino-2-ethyl)-9-hydroxyellipticinium chloride) with these retinoids was studied.

Material and Methods:

The ex vivo cell viability inhibition by BA016DD537 in the presence of the retinoids 13CRA and ATRA was tested against B16BL6 melanoma cell line using the 3-(4,5 dimethylthiazol-2-yl)-2,5-diphenyl-2H-tetrazolium bromide (MTT) calorimetric assay.

About 1000 cells were seeded in a 96-well culture plate 24 hours before adding increasing concentrations of BA016DD537, from 1 pM to 100 pM, in the absence or in the presence of 10 nM of retinoids. The plate was incubated at 37° C. for 3 days. 10 μl MTT stock solution (5 mg/ml in phosphate buffer saline) was added to 90 μl of complete medium in each well, the incubation was continued for 3 h at 37° C. 100 μl of lysis buffer (20% sodium dodecyl sulfate, 10 mM HCl, 1×PBS) was added to each well, and the plate was incubated overnight. The absorbance was determined using an Integrated EIA Management System (Labsystem) at a wavelength of 570 nm.

Results

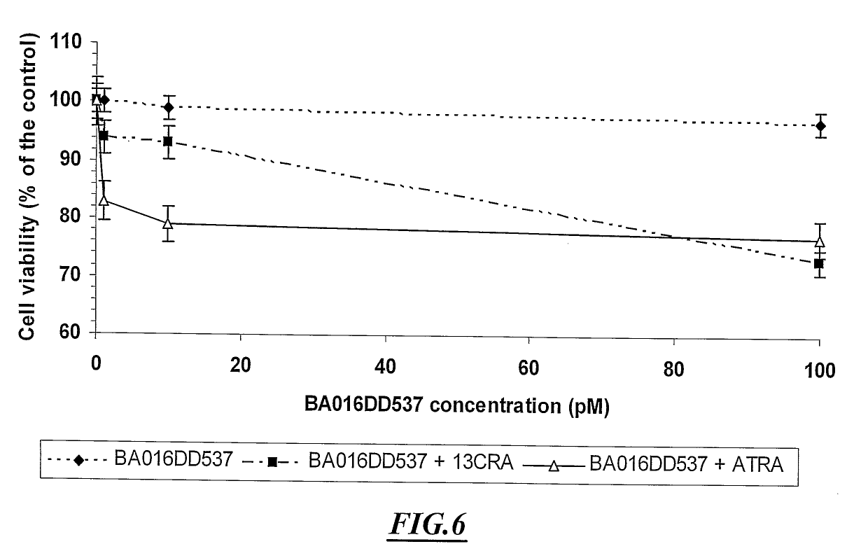

The B16BL6 invasive melanoma cells display a high invasive phenotype. The aim of synergy assay was to inhibit the tumoral cell viability at the lowest concentrations of BA016DD537. No cell viability inhibition was observed in the presence of 10 nM of the retinoids 13CRA and ATRA, only. Cell treatment with lower doses of BA016DD537 in the presence of 10 nM retinoids results in increased tumoral cell viability inhibition (FIG. 6).

At the same time activity of BA016DD537 at the lowest concentrations in the absence of retinoids was not observed. The efficacy of BA016DD537 at 100 nM was the same as at 1 pM in the presence of ATRA 10 nM. Thus the dose of BA016DD537 can be decreased 100 000-fold, when used in combination with retinoids, to obtain the same results than when used in absence of retinoids.

### Example 11

### Comparison of Biological Activity of Two 9-Hydroxy Ellipticine Derivates: Monomesylate and Bimesylate

Antitumor activity of two ellipticine derivates, BA016FZ539 (2-(beta piperidino-2-ethyl)-9-hydroxyellipticinium methanesulfonate, or “monomesylate”) and the corresponding bimesylate derivative

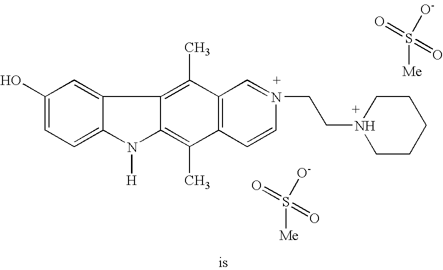

(hereafter “bimesylate”), were assessed using two independent experiments. Inhibition of colonies formation in semi-solid medium was first evaluated with the cloning assay on the murine melanoma cell line B16F10.

Secondly, cell proliferation of two human pancreatic cell lines (MIA PaCA-2 and PANC1) and one murine melanoma cell line (B16F10) were quantified in presence of monomesylate and bimesylate using the SRB and MTT tests.

Material and Methods:

Cloning Assay

Cells were embedded in complete culture medium supplemented with 0.8% methyl-cellulose (Methocel MC4000, Sigma), seeded in triplicate into 35-mm dishes and incubated at 37° C. in a humidified 5% CO₂ atmosphere. The number of cells seeded was 1000 cells per dish. After 9 days, macroscopic clones of murine melanoma cell line B16F10 were counted.

MTT and SRB Tests

Growth studies were performed using both MTT and SRB calorimetric assay (Sigma). About 1500 B16F10 or 3000 pancreatic cells (MIA PaCa-2 and PANC1) were seeded in a 96-well culture plate before adding increasing concentrations of monomesylate or bimesylate.

The plates were incubated at 37° C. for 3 days and then were treated depending on SRB or MTT protocols (see material and methods).

In these two cases, the proliferation rates were calculated from the OD readings using the untreated cells as 100%.

Results:

TABLE 11
Inhibition of cell proliferation

| | IC50 (SRB Test-72 h) | | IC50 (MTT Test-72 h) | |
|---|---|---|---|---|
| | Mono- | | Mono- | |
| Cell lines | mesylate | Bimesylate | mesylate | Bimesylate |
| B16F10 | 4.3 μM | 1.8 μM | 2 μM | 2.8 μM |
| Mia Paca-2 | 5.6 μM | 2.5 μM | nd | nd |
| PANC-1 | 50.4 μM | 37.6 μM | nd | nd |

Nd: not determined

B16F10 was tested both with SRB and MTT assays whereas PANC1 was investigated only with SRB assay. There were no significant differences between the IC50 of monomesylate and bimesylate.

TABLE 12
Inhibition of B16F10 colony formation

| | B16F10 Inhibition of colony formation |
|---|---|
| Monomesylate | IC50 = 67 nM |
| Bimesymate | IC50 = 21 nM |

Both Monomesylate and bimesylate showed similar 50% inhibitory concentration (IC50) on colony formation in semi-solid medium, respectively 67 and 21 nM.

Marked inhibitory effects on the growth of invasive murine melanoma cell line B16F10 were effectively obtained in methyl-cellulose in presence of these two drugs.

Our results confirmed also that inhibition of colony formation occurs at non-proliferative concentration as measured using the MTT test (Table 12).

In conclusion, monomesylate and bimesylate possess the same biological activity regarding results from cloning assay and cell proliferation tests.

Together these data strongly suggest that the ellipticine derivates have potential for the development as an anti-tumoral agent.

1. Use of a 9-hydroxy ellipticine derivative of formula (III): 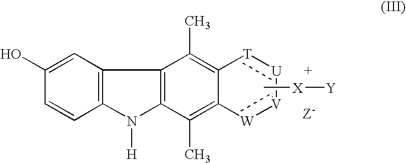
optionally in the form of an acid addition salt,
wherein
X is an alkyl group having 2 or 3 carbon atoms, optionally branched, and optionally substituted by OH, NRR′, CN, OR, COOR, wherein R and R′ are independently H or a C1-C4 alkyl group;
Y is —NR1R2, wherein R1 and R2 are independently H or a C1-C6 alkyl group, or R1 and R2 form together with the N atom, to which they are attached, a saturated or unsaturated 5- or 6-membered heterocycle, wherein —NR1R2 may be in the form of a quaternary ammonium salt resulting from the addition of a pharmaceutically acceptable mineral or organic acid, so that the compound of formula (I) is in the form of an acid addition salt;
or Y is a benzyl, a phenyl or a C5 or C6 aryl or 5- or 6-heteroaryl group; and
Z⁻ is an anion of a pharmaceutically acceptable mineral or organic acid;
the -X-Y side chain is attached to either T, U, V or W as appropriate;
T, U, V and W are either a C atom or a N atom, so as to form a pyridyl ring and the remaining T, U, V and/or W are C atoms,
provided that the -X-Y side chain is attached to the one of T, U, V and W being a N atom,
it being understood that [symbol] represents either a single bond or a double bond, as appropriate, so that the system formed with the fused pyridyl ring is aromatic and the resulting cation 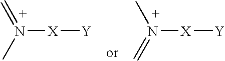
is formed,
for the manufacture of a medicament intended for the treatment of cancer.

2. The use according to claim 1, wherein said 9-hydroxy ellipticine derivative has the formula (IV): 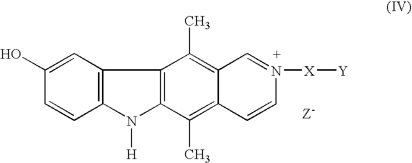
optionally in the form of an acid addition salt,
wherein X, Y and Z⁻ are as defined in claim 1.

3. The use according to claim 1, wherein X is ethyl or propyl.

4. The use according to claim 1, wherein Y is —NR1R2 and each of R1 and R2 is an ethyl group, wherein —NR1R2 may be in the form of a quaternary ammonium salt resulting from the addition of a pharmaceutically acceptable mineral or organic acid, so that the compound of formula (I) is in the form of an acid addition salt.

5. The use according to claim 1, wherein Y is selected from the group consisting of piperidine, pyrrolidinyl, pyridine and pyrimidine, and their quaternary ammonium salts.

6. The use according to claim 1, wherein said 9-hydroxy ellipticine derivative is 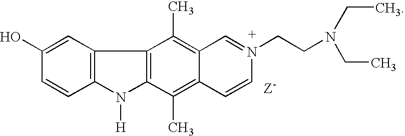
or its resulting quaternary ammonium salts.

7. The use according to claim 1, wherein said 9-hydroxy ellipticine derivative is 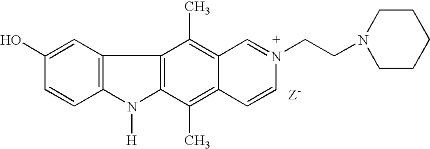
or its resulting quaternary ammonium salts.

8. The use according to claim 1, wherein Z⁻ is methanesulfonate.

9. The use according to claim 1, wherein said 9-hydroxy ellipticine derivative is:

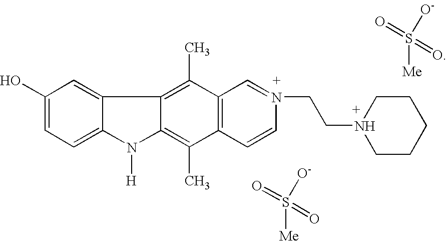

10. The use according to claim 1, wherein the medicament is intended for reversing the transformed phenotype of a tumor cell.

11. The use according to claim 1, wherein said tumor cell is characterized by an invasive phenotype.

12. The use according to claim 1, wherein said medicament is intended for the treatment of metastasis.

13. The use according to claim 1, wherein said medicament is intended for the treatment of cancer in a subject escaping cytotoxic chemotherapy.

14. The use according to claim 1, wherein said medicament is administered in combination with a differentiating agent.

15. The use according to claim 14, wherein said differentiating agent is selected from the group consisting of vitamin A and its synthetic analogs, retinoids, vitamin D and its analogs, and peroxisome proliferator-activated receptors (PPAR) ligands.

16. A pharmaceutical composition comprising a 9-hydroxy ellipticine derivative of formula (III) or (IV) as defined in claim 1, and a differentiating agent, in a pharmaceutically acceptable carrier.

17. The pharmaceutical composition according to claim 16, wherein said differentiating agent is selected from the group consisting of vitamin A and its synthetic analogs, retinoids, vitamin D and its analogs, and peroxisome proliferator-activated receptors (PPAR) ligands.

18. A product comprising a 9-hydroxy ellipticine derivative of formula (III) or (IV) as defined in claim 1, and a differentiating agent, as a combined preparation for simultaneous, separate or sequential use for the treatment of cancer.

19. A product according to claim 18, as a combined preparation for simultaneous, separate or sequential use for reversing the transformed phenotype of a tumor cell.

20. The product according to claim 18, wherein said differentiating agent is selected from the group consisting of vitamin A and its synthetic analogs, retinoids, vitamin D and its analogs, and peroxisome proliferator-activated receptors (PPAR) ligands.

21. A 9-hydroxy ellipticine derivative of formula (III): 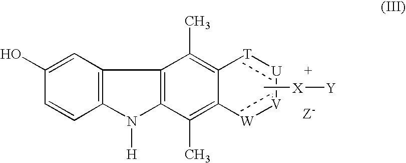
optionally in the form of an acid addition salt,
wherein
X is an alkyl group having 2 or 3 carbon atoms, optionally branched, and optionally substituted by OH, NRR′, CN, OR, COOR, wherein R and R′ are independently H or a C1-C4 alkyl group;
Y is —NR1R2, wherein R1 and R2 are independently H or a C1-C6 alkyl group, or R1 and R2 form together with the N atom, to which they are attached, a saturated or unsaturated 5- or 6-membered heterocycle, wherein —NR1R2 may be in the form of a quaternary ammonium salt resulting from the addition of a pharmaceutically acceptable mineral or organic acid, so that the compound of formula (I) is in the form of an acid addition salt;
or Y is a benzyl, a phenyl or a C5 or C6 aryl or 5- or 6-heteroaryl group; and
Z⁻ is an anion of a pharmaceutically acceptable mineral or organic acid;
the -X-Y side chain is attached to either T, U, V or W as appropriate;
T, U, V and W are either a C atom or a N atom, so as to form a pyridyl ring and the remaining T, U, V and/or W are C atoms,
provided that the -X-Y side chain is attached to the one of T, U, V and W being a N atom,
it being understood that [symbol] represents either a single bond or a double bond, as appropriate, so that the system formed with the fused pyridyl ring is aromatic and the resulting cation 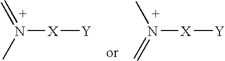
is formed,
with the proviso that said 9-hydroxy ellipticine derivative is not 2-(diethylamino-2-ethyl)-9-hydroxyellipticinium chloride, 2-(diethylamino-2-ethyl)-9-hydroxyellipticinium acetate, 2-(diisopropylamino-ethyl)-9-hydroxyellipticinium acetate, or 2-(beta piperidino-2-ethyl)-9-hydroxyellipticinium acetate.

22. The 9-hydroxy ellipticine derivative according to claim 21, said 9-hydroxy ellipticine derivative has the formula (IV): 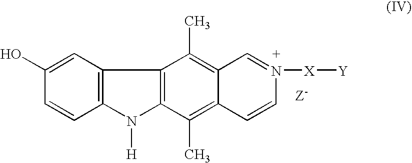
optionally in the form of an acid addition salt,
wherein X, Y and Z⁻ are as defined in claim 21, and
with the proviso that said 9-hydroxy ellipticine derivative is not 2-(diethylamino-2-ethyl)-9-hydroxyellipticinium chloride, 2-(diethylamino-2-ethyl)-9-hydroxyellipticinium acetate, 2-(diisopropylamino-ethyl)-9-hydroxyellipticinium acetate, or 2-(beta piperidino-2-ethyl)-9-hydroxyellipticinium acetate.

23. The 9-hydroxy ellipticine derivative according to claim 22 wherein X is ethyl and Y is piperidine, with the proviso that said 9-hydroxy ellipticine derivative is not 2-(beta piperidino-2-ethyl)-9-hydroxyellipticinium acetate.

24. The 9-hydroxy ellipticine derivative according to claim 21 which is 2-(beta piperidino-2-ethyl)-9-hydroxyellipticinium methanesulfonate or its resulting quaternary ammonium salts.

25. The 9-hydroxy ellipticine derivative according to claim 21 which is:

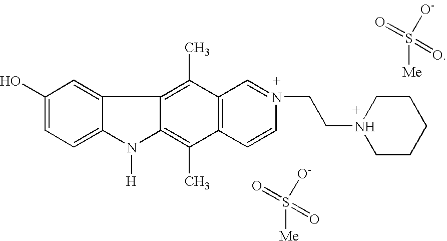

26. The 9-hydroxy ellipticine derivative according to claim 21 or 22 which is 2-(diethylamino-2-ethyl)-9-hydroxyellipticinium methanesulfonate or its resulting quaternary ammonium salts.

27. A pharmaceutical composition comprising a 9-hydroxy ellipticine derivative according to claim 21, in a pharmaceutically acceptable carrier.
